(19) United States
(12) Patent Application Publication
(10) Pub. No.: US 2026/0171203 A1
(43) Pub. Date: Jun. 18, 2026

(54) MONITORING WELLNESS USING PERSONALIZED MODELS

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US)

(21) Appl. No.: 19/419,406

(22) Filed: Dec. 15, 2025

Related U.S. Application Data
(60) Provisional application No. 63/733,645, filed on Dec. 13, 2024.

(51) Int. Cl. G16H 20/00; G06N 3/044; G06N 3/0475; G16H 10/60; G16H 40/67; G16H 50/70

(52) U.S. Cl. CPC G16H 20/00; G06N 3/044; G06N 3/0475; G16H 10/60; G16H 40/67; G16H 50/70

(57) ABSTRACT

Systems and methods are provided for monitoring wellness of a user. A parameter interface that receives a time series of values for each of a plurality of parameters representing a subject. A global model, trained on data from a plurality of subjects, receives the time series of values for each of a first subset of the plurality of parameters. A personalized model, trained only on data from the subject, receives the time series of values for each of a second, different, subset of the plurality of parameters. A model selector selects one of the global model and the personalized model according to the time series of values for each of a third subset of the plurality of parameters. An output of the selected one of the global model and the personalized model is provided to a subject interface.

### RELATED APPLICATION

This application claims priority from U.S. Application No. 63/733,645, filed 13 Dec. 2024. The subject matter of this application is incorporated herein by reference in its entirety.

### TECHNICAL FIELD

This invention relates to health information technology, and more particularly, to monitoring wellness using personalized models.

### BACKGROUND

Many factors affecting the health and wellness of an individual can be initially subtle and difficult to detect. Early detection of these factors can allow for effective intervention before the health and wellness of the individual is negatively impacted. In addition, it is often difficult to detect early signs of diseases or disorders that may impact the capacity of an individual for a given task or activity. In general, determinations of an individual's capacity must be performed on measurable symptoms or self-reporting, which can allow subtle decreases in capacity to go unnoticed.

### SUMMARY

In one example, a system includes a processor and a non-transitory computer-readable medium storing executable instructions that are executable by the processor. The executable instructions include a parameter interface that receives a time series of values for each of a plurality of parameters representing a subject. A global model, trained on data from a plurality of subjects, receives the time series of values for each of a first subset of the plurality of parameters. A personalized model, trained only on data from the subject, receives the time series of values for each of a second subset of the plurality of parameters. The first subset of the plurality of parameters is different from the second subset of the plurality of parameters. A model selector selects one of the global model and the personalized model according to the time series of values for each of a third subset of the plurality of parameters. An output of the selected one of the global model and the personalized model is provided to a subject interface.

In another example, a method is provided for monitoring a wellness of a user. A time series of values are received for each of a plurality of parameters representing a subject. Either a global model, trained on data from a plurality of subjects, or a personalized model, trained only on data from the patient, is selected. The global model receives the time series of values for each of a first subset of the plurality of parameters and a personalized model, and the personalized model receives the time series of values for each of a second, different subset of the plurality of parameters. The selection of the models can be made according to the time series of values for each of a third subset of the plurality of parameters. An output of the selected one of the global model and the personalized model is provided to a user interface.

In a further example, a system includes a processor, a network interface, and a non-transitory computer-readable medium storing executable instructions that are executable by the processor. The executable instructions include a parameter interface that receives a time series of values for each of a plurality of parameters representing a subject through an Internet connection via the network interface. A first recurrent neural network is trained on a plurality of training samples from a plurality of subjects. Each of the training samples include a value representing an outcome for a subject of the plurality of subjects and a time series of values for each of a first subset of the plurality of parameters. A second recurrent neural network is trained on a plurality of training samples from the subject. Each of the training samples includes a value representing an outcome for the subject and a time series of values for each of a second subset of the plurality of parameters. The first subset of the plurality of parameters are different from the second subset of the plurality of parameters.

A model selector selects one of the first recurrent neural network and the second recurrent neural network according to the time series of values for each of a third subset of the plurality of parameters, provides the time series of values for a subset of the plurality of parameters to the selected one of the first recurrent neural network and the second recurrent neural network as inputs, and provides an output of the selected one of the first recurrent neural network and the second recurrent neural network to a user interface, the output of the selected one of the first recurrent neural network and the second recurrent neural network including a value representing a likelihood that the subject will experience symptoms associated with a condition within a predetermined period of time.

### BRIEF DESCRIPTION OF THE DRAWINGS

FIG. 1 illustrates one example of a system for monitoring a health and wellness of a subject;

FIG. 2 illustrates another example of a system for monitoring wellness of a subject;

FIG. 3 is a first screenshot from the physician dashboard illustrating data for a population of patients;

FIG. 4 is a second screenshot from the physician dashboard illustrating data for a population of patients;

FIG. 5 is a first screenshot from the physician dashboard illustrating data for an individual patient;

FIG. 6 is a second screenshot from the physician dashboard illustrating data for an individual patient;

FIG. 7 is a third screenshot from the physician dashboard illustrating data for an individual patient;

FIG. 8 is a screenshot for an interface in the physician dashboard that allows a physician to selecting personalized thresholds for a patient for various metrics provided by instantiations of the global model and the personalized model;

FIG. 9 illustrates a method for monitoring a wellness of a user; and

FIG. 10 is a schematic block diagram illustrating an exemplary system of [0017] hardware components capable of implementing examples of the systems and methods disclosed in FIGS. 1-9.

### DETAILED DESCRIPTION

The term “wellness” as used herein in intended to refer to the mental, physical, cognitive, behavioral, social, and emotional health of a subject and should be construed to cover each of the health, function, balance, resilience, homeostasis, disease, and condition of the subject. In various examples herein, the wellness of the subject can be related to the readiness of the subject to perform job-related, athletic, or everyday functions, enter into a flow or zone state, the susceptibility of the subject to an infectious disease, the ability of the subject to resist the effects of addiction, worsening pain of a subject, increased or reduces stress, anxiety, a quality of life of the subject, and similar qualities of a subject.

A “wellness-relevant parameter” is a parameter that is relevant to the wellness of a subject.

A “biological rhythm” is any chronobiological phenomenon that affects human beings, including but not limited to, circadian rhythms, ultradian rhythms, infradian rhythms, diurnal cycle, sleep/wake cycles, and patterns of life.

A “neuromodulation technique or mode,” or “neuromodulation” as [0021] described herein, is any suitable technique that applies localized energy (or other mode of neuromodulation) to the brain for the purpose of modulating (e.g., activating, inhibiting, regulating, resetting, normalizing) neural activity and neural networks or altering the permeability of the blood-brain barrier for an applied therapeutic at a specific location. Non-limiting neuromodulation techniques or modes include ultrasound such as, for example, focused ultrasound; electrical such as, for example superficial (including transcutaneous, percutaneous or subcutaneous stimulation) or cortical or deep brain stimulation, magnetic stimulation including, for example transcranial magnetic stimulation; optogenetic stimulation; or application of pulses of electromagnetic radiation. Other modes of neuromodulation include application of light, pressure, and heat/cold. As used herein, the term “modulation” refers to inhibiting, exciting, modulating, regulating, resetting or normalizing neural activity.

A “focused ultrasound treatment”” as described herein, is any treatment that applies ultrasound to the brain for the purpose of neuromodulation and modulating neural activity or disrupting the blood-brain barrier for the purpose of allowing targeted entry of therapeutics into the brain. Accordingly, a focused ultrasound treatment can involve the use of ultrasound to modulate neural activity, the use of ultrasound in combination with microbubbles to disrupt the blood-brain barrier and allow selective entry of an appropriate therapeutic into the brain, or a combination of blood-brain barrier disruption with neuromodulation of appropriate targets. It will be appreciated that the use of focused ultrasound for disrupting the blood-brain barrier will involve a degree of neuromodulation due to the impact of the ultrasound on the surrounding tissue, but references to a focused ultrasound treatment comprising a combination of neuromodulation and disruption of the blood-brain barrier refers to at least two separate applications of focused ultrasound, with a first application of focused ultrasound targeted at a location at which penetration of therapeutics into the brain is desired and a second application of focused ultrasound at a location with the brain tissue for which neuromodulation is desired.

A “portable monitoring device,” as used herein, refers to a device that is worn by, carried by, or implanted within a subject that incorporates either or both of an input device and user interface for receiving input from the user and sensors for monitoring either a wellness-relevant parameter or a parameter that can be used to calculate or estimate a wellness-relevant parameter.

An “index”, as used herein, is intended to cover composite statistics derived from a series of observations and used as an indicator or measure. An index can be an ordinal, continuous, or categorical value representing the observations and correlations, and should be read to encompass statistics traditionally referred to as “scores” as well as the more technical meaning of index.

A “clinical parameter”, as used herein, can be any continuous or categorical parameter representing the mental, physical, cognitive, behavioral, social, and emotional health of a subject and can represent any or all of the health, function, a zone or flow state, balance, resilience, homeostasis, disease, and condition of the subject.

A “continuous parameter,” as used herein, is used broadly to refer to a parameter that can assume any value within a predefined range to a predetermined level of precision. Accordingly, a value is referred to herein as a continuous parameter even if limited, in practice, to a finite number of discrete values within the range by the resolution at which the value is measured, calculated, or stored.

An “physiological sensing device,” as used herein, is a device to measure one or more physiological parameters and/or biological rhythms. A physiological sensing device is often implanted, ingested, or wearable, although in some instances an off-body device can be used to capture physiological parameters.

A “portable computing device,” as used herein, is a computing device that can carried by the subject, such as a smartphone, smart watch, tablet, notebook, and laptop, that can measure a wellness-relevant parameter either through sensors on the device or via interaction with the subject. A portable computing device can include, for example, a user interface for receiving an input from the subject, kinematic sensors for measuring activity by the subject, and location services that track a location of the subject.

As used herein, a “predictive model” is a mathematical model or machine learning model, implemented as machine readable instructions stored on a tangible medium and executed by an associated processor, that either predicts a future state of a parameter or estimates a current state of a parameter that cannot be directly measured.

A “server” as used herein, is a computing device that is in communication with one or more other computing devices via a network connection. Accordingly, as used herein, such as device includes at least a non-transitory computer readable medium storing executable instructions, a processor operatively connected to the medium to execute the instructions, and a network interface that allows the server to communicate over the network connection.

As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

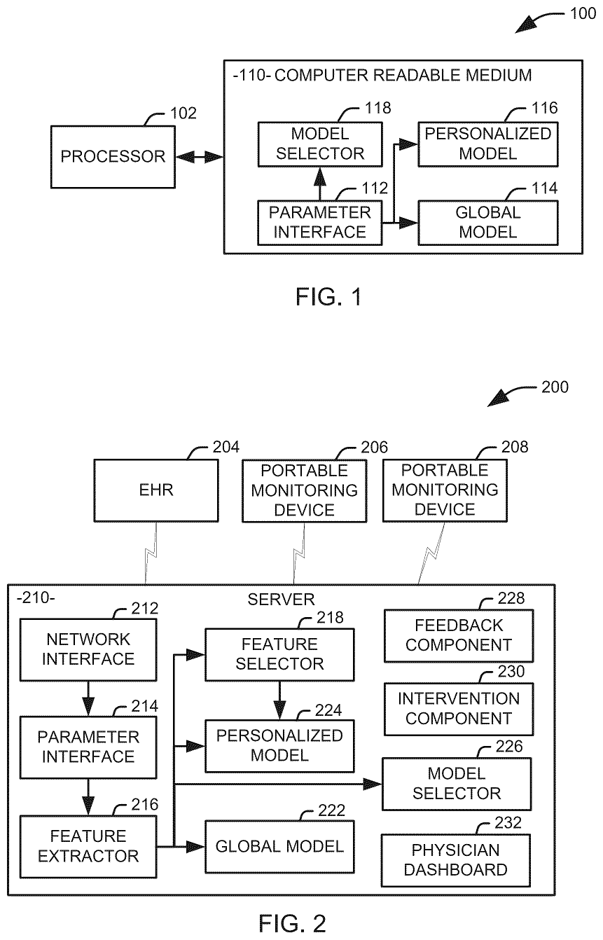

FIG. 1 illustrates one example of a system 100 for monitoring a health and wellness of a subject. The system 100 includes a processor 102 and a non-transitory computer-readable medium 110 storing executable instructions that are executable by the processor. The executable instructions include a parameter interface 112 that receives a time series of values for each of a plurality of parameters representing a subject. It will appreciated that the parameter interface 112 can receive parameters from any or all of an electronic health records database, devices carried by the subject, devices implanted in the subject, and devices worn by the patient, each providing parameters to the system 100 via a local or wide-area network connection. Examples of appropriate parameters can include, for example, physiological parameters, cognitive parameters, motor or musculoskeletal parameters, sensory parameters, parameters related to sleep, biomarker parameters, and sociobehavioral parameters.

A global model 114 is trained on data from a plurality of subjects. The global model 114 receives the time series of values for each of a first subset of the plurality of parameters. It will be appreciated that the first subset of the plurality of parameters can be a proper subject of the plurality of parameters or can include all of the plurality of parameters. A personalized model 116 is trained only on data from the subject. The personalized model 116 receives the time series of values for each of a second subset of the plurality of parameters. Like the global model, the second subset of the plurality of parameters can be a proper subject of the plurality of parameters or can include all of the plurality of parameters, but in the illustrated example, the first subset of the plurality of parameters is different from the second subset of the plurality of parameters. It will be appreciated that each of the global model 114 and the personalized model 116 can be implemented as any appropriate model, and in one example, each of the global model and the personalized model can be implemented as recurrent neural networks.

In one example, each of the global model 114 and the personalized model 116 provide an output representing an overall wellness of the subject. In other implementations, the output can represent a “sphere” or aspect of wellness, such as sleep quality, autonomic function, stress, or similar values. The output of the global model 114 and the personalized model 116 can be an index representing the wellness of the patient or a categorical output representing a degree of wellness (e.g., excellent, good, fair, poor). The global model 114 and the personalized model 116 can also represent a likelihood that the subject has or is at risk for a specific disorder or class of disorders. This can be expressed as a continuous value that is related to a probability or risk of the disorder or as a categorical value representing ranges of probability or risk for the disorder.

A model selector 118 selects one of the global model 114 and the personalized model 116 according to the time series of values for each of a third subset of the plurality of parameters, an output of the selected one of the global model and the personalized model being provided to a user interface. In some implementations, the third subset of the plurality of parameters can be a subset of the second subset of the plurality of models. In these implementations, the model selector 118 can review the incoming time series according to an appropriate quality metric, with the personalized model 116 selected when the data is of sufficient quality to support analysis at the personalized model. The quality metric can represent, for example, the completeness of data (e.g., absence of missing data points in the time series), the feasibility of data (e.g., absence of data points that are either impossible or extraordinarily unlikely), or metadata provided by one or more data sources, such as sensors, that indicate possible errors at the data source. The global model 114 can be automatically selected when insufficient training data has been collected for the personalized model 116, for example, when the subject initially begins using the system 100. The output of the selected model can be stored at the non-transitory computer medium 110 or on another computer readable medium or displayed to the subject or another user of the system, such as a physician, counselor, technician, coach, or employer at an associated display.

It will be appreciated that additional global and personalized models (not shown) can be included for other outputs, such as other specific disorders or aspects of wellness. In these implementations, it will be appreciated that the specific inputs, that is, the subset of the plurality of features used by the model, will differ among the global models for various outputs and the personalized models for various outputs, as well as between pairs of global and personalized models of a given output. For each output, the model selector 118 selects between the personalized model and the global model for the output based on at least a subset of the plurality of parameters. It will be appreciated that these determinations can be made independently, such that a global model may be selected for a first output while a personalized model is selected for a second output.

Further, the parameters predicted by a given model can be used as inputs to another model. For example, a predicted level of anxiety of a patient in a predetermined window of time can be relevant to a prediction of stress in that time frame, a prediction of substance use during that period, or a likelihood of cardiac issues. It will be appreciated that the model selector 118 can select these constituent models independently, such that a first model predicting a parameter used in a second model can use one of the personalized and the global model for that model, while the second model can use the other.

FIG. 2 illustrates another example of a system 200 for monitoring wellness of a subject. The system 200 includes a plurality of data sources 204, 206, and 208 that provide data to a central server 210. The plurality of data sources 204, 206, and 208 include an electronic health records (EHR) database 204 that can be accessed by the central server 210. The data sources can also include portable monitoring devices 206 and 208 that include sensors for monitoring systems tracking wellness-relevant parameters for the user. It will be appreciated that a given portable monitoring device (e.g., 206) can either communicate directly with the central server 210 to provide the wellness-relevant parameters to the server or with another portable monitoring device (e.g., 208) that relays the wellness-relevant parameters to the server. In one example, the plurality of portable monitoring devices can include a physiological sensing device, implemented, for example, as a worn or implanted device, and a portable computing device. Additional parameters can be either retrieved from the EHR database 204 and/or other available databases via a network interface 212 associated with the server 210. These parameters can include, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history.

A plurality of parameters can be received from the electronic health database 204, and the portable monitoring systems 206 and 208. Parameters that can be retrieved from an electronic health records (EHR) interface and/or other available databases, include, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history. Parameters can also be measured via diagnostic systems, imaging systems, wearable sensors, cognitive tests, questionnaires, and other means. As noted above, wellness-relevant parameters can include at least physiological, cognitive, motor/musculoskeletal, sensory, sleep, biomarkers and behavioral parameters. Table I provides non-limiting examples of physiological parameters that can be measured and exemplary tests, devices, and methods, to measure the physiological parameters.

TABLE I

| | Exemplary Devices and Methods to Measure |
|---|---|
| Physiological Parameter | Physiological Parameters |
| Brain Activity | Electroencephalogram, Magnetic Resonance |
| | Imaging, including functional Magnetic Resonance |
| | Imaging (fMRI), PET, SPECT, MEG, near-infrared |
| | spectroscopy, functional near-infrared spectroscopy, |
| | and other brain imaging modalities looking at |
| | electrical, blood flow, neurotransmitter, and |
| | metabolic MRI, taken either during a cognitive task |
| | or while the patient is at rest |
| Brain Structure | Magnetic Resonance Imaging |
| Heart rate | Electrocardiogram and Photoplethysmogram |
| Heart rate variability | Electrocardiogram, Photoplethysmogram |
| Eye tracking | Pupillometry, including tracking saccades, fixations, |
| | and pupil size (e.g., dilation) |
| Perspiration | Perspiration sensor |
| Retinal | OCT and angiography |
| Retinal anatomy, layers, | Other Retinal imaging including wide field |
| retinal vasculature | |
| Blood pressure | Sphygmomanometer |
| Body temperature | Thermometer, infrared thermography |
| Blood oxygen saturation | Pulse oximeter/accelerometer |
| and respiratory rate | |
| Skin conductivity | Electrodermal activity |
| Facial emotions | Camera or EMG based sensors for emotion and |
| | wellness |
| Sympathetic and | Derived from the above measurements |
| parasympathetic tone | |

The physiological parameters can be measured in clinical settings with appropriate devices or in non-clinical settings via wearable, implantable, or portable devices. Some information can also be determined from self-reporting by the user via applications in a mobile device or via interaction with applications on the mobile device. For example, a smart watch, ring, or patch can be used to measure the user's heart rate, heart rate variability, body temperature, blood oxygen saturation, movement, and sleep. In a non-clinical setting, these values can also be subject to a diurnal analysis to estimate variability. Eye tracking can be performed, for example, using a camera on a mobile device and specialized software.

Table Il provides non-limiting examples of cognitive parameters that are gamified and that can be measured and exemplary methods and tests/tasks to measure such cognitive parameters. The cognitive parameters can be assessed by a battery of cognitive tests that measure, for example, executive function, decision making, working memory, attention, and fatigue.

TABLE II

| | Exemplary Tests and Methods to Measure |
|---|---|
| Cognitive Parameter | Cognitive Parameters |
| Temporal discounting | Kirby Delay Discounting Task |
| Alertness and fatigue | Psychomotor Vigilance Task |
| Focused attention and | Erikson Flanker Task |
| response inhibition | |
| Working memory | N-Back Task |
| Attentional bias towards | Dot-Probe Task |
| emotional cues | |
| Inflexible persistence | Wisconsin Card Sorting Task |
| Decision making | Iowa Gambling Task |
| Risk taking behavior | Balloon Analogue Risk Task |
| Inhibitory control | Anti-Saccade Task |
| Sustained attention | Sustained Attention |
| Executive function | Task Shifting or Set Shifting Task |
| Long term memory | Identifying pictures of famous people and other |
| | memory related tasks |

These cognitive tests can be administered in a clinical/laboratory setting or in a naturalistic, non-clinical setting such as when the user is at home, work or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch. In one example, the patient can be allowed to explore a virtual reality environment and collect items within the environment. The patient is then asked to recount where each item was found within the virtual environment and the relationship of that location to a starting point, testing the patient's ability to recall spatial relationships among the virtual locations.

TABLE III provides non-limiting examples of parameters associated with movement and activity of the user, referred to herein alternatively for ease of reference as “motor parameters,” that can be measured and exemplary tests, devices, and methods. The use of portable monitoring, physiological sensing, and portable computing devices allows the motor parameters to be measured. Using embedded accelerometer, GPS, and cameras, the user's movements can be captured and quantified to see how wellness affects them and related to the wellness-relevant parameters. Range of motion and gait analysis can be performed in a clinical setting using appropriate motion capture and camera equipment for evaluation.

TABLE III

| Motor/ | |
|---|---|
| Musculoskeletal | Exemplary Tests and Methods to Measure |
| Parameter | Motor/Musculoskeletal Parameters |
| Activity level | Daily movement total, time of activities, from |
| | wearable accelerometer, steps, Motion Capture |
| | data, gait analysis, GPS, deviation from established |
| | geolocation patterns, force plates |
| Gait analysis | Gait mat, camera, force plats |
| Range of motion | Motion capture, camera, |

TABLE IV provides non-limiting examples of parameters associated with sensory acuity of the user, referred to herein alternatively for ease of reference as “sensory parameters,” that can be measured and exemplary tests, devices, and methods.

TABLE IV

| | Exemplary Tests and Methods to Measure |
|---|---|
| Sensory Parameter | sensory Parameters |
| Vision | Visual acuity test, visual field tests, eye tracking, |
| | EMG |
| Hearing | Hearing tests |
| Touch | Two-point discrimination, frey filament |
| Smell/taste | |
| Vestibular | Vestibula function test |

TABLE V provides non-limiting examples of parameters associated with a sleep quantity, phases, and quality of the user, referred to herein alternatively for ease of reference as “sleep parameters,” that can be measured and exemplary tests, devices, and methods.

TABLE V

| Sleep | Exemplary Tests and Methods to Measure Sleep |
|---|---|
| Parameter | Parameters |
| Sleep from | Sleep onset & offset, sleep quality, sleep quantity, |
| wearables | from wearable accelerometer, temperature, and |
| | PPG, |
| Sleep Questions | Pittsburg Sleep Quality Index, Functional Outcomes |
| | of Sleep Questionnaire, Fatigue Severity Scale, |
| | Epworth Sleepiness Scale |
| Devices | Polysomnography; ultrasound, camera, bed |
| | sensors, EEG |
| Circadian Rhythm | Light sensors, actigraphy, serum levels, core body |
| | temperature |

TABLE VI provides non-limiting examples of parameters extracted by locating biomarkers associates with the user, referred to herein alternatively for ease of reference as “biomarker parameters,” that can be measured and exemplary tests, devices, and methods. Biomarkers can also include imaging and physiological biomarkers related to a state of chronic wellness and improvement or worsening of the chronic wellness state.

TABLE VI

| | Exemplary Tests and Methods to Measure |
|---|---|
| Biomarkers Parameter | Biomarkers Parameters |
| Genetic biomarkers | Genetic testing |
| Immune biomarkers | Blood, saliva, and/or urine tests |
| including TNF-alpha, | |
| immune alteration (e.g., | |
| ILs), oxidative stress, and | |
| hormones (e.g., cortisol) | |

TABLE VII provides non-limiting examples of psychosocial and behavioral parameters, referred to herein alternatively for ease of reference as “psychosocial parameters,” that can be measured and exemplary tests, devices, and methods.

TABLE VII

| Psychosocial or | Exemplary Tests and Methods to Measure |
|---|---|
| Behavioral Parameter | Psychosocial or Behavioral Parameters |
| Symptom log | Presence of specific symptoms (i.e., fever, headache, |
| | cough, loss of smell) |
| Medical Records | Medical history, prescriptions, setting for treatment |
| | devices such as spinal cord stimulator, imaging data |
| Wellness Rating | Visual Analog Scale, Defense & Veterans wellness |
| | rating scale, wellness scale, Wellness Assessment |
| | screening tool and outcomes registry |
| Burnout | Burnout inventory or similar |
| Physical, Mental, and | User-Reported Outcomes Measurement Information |
| Social Health | System (PROMIS), Quality of Life Questionnaire |
| Depression | Hamilton Depression Rating Scale |
| Anxiety | Hamilton Anxiety Rating Scale |
| Mania | Snaith-Hamilton Pleasure Scale |
| Mood/ | Profile of Mood States; Positive Affect Negative Affect |
| Catastrophizing scale | Schedule |
| Affect | Positive Affect Negative Affect Schedule |
| Impulsivity | Barratt Impulsiveness Scale |
| Adverse Childhood | Childhood trauma |
| Experiences | |
| Daily Activities | Exposure, risk taking |
| Daily Workload and Stress | NASA Task Load Index, Perceived Stress Scale |
| | (PSS), |
| | Social Readjustment Rating Scale (SRRS) |
| Social Determents of | Social determents of health questionnaire |
| Health | |

The behavioral and psychosocial parameters can measure the user's functionality as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue. This affect detection can be performed using an appropriate predictive model trained on faces of users mimicking or experiencing a given emotion or mental state.

Additional elements of monitoring can include the monitoring of the user's compliance with the use of a smart phone, TV, portable device, a portable device. For example, a user may be sent messages by the system inquiring on their wellness level, general mood, or the status of any other wellness-relevant parameter on the portable computing device. A measure of compliance can be determined according to the percentage of these messages to which the user responds via the user interface on the portable computing device and used as an indication as to how likely the patient is to comply with treatment protocols and monitoring.

Many behavioral and psychosocial parameters can be determined from the user's functionality and activity as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. One of the portable monitoring systems 206 and 208 can be a smart device, such as a smartphone, tablet, or personal computer that can be used to administer the subjective/self-reporting questionnaires. Using embedded accelerometers and cameras, these smart devices can also be used to capture the facial expression analysis to analyze the user's facial expressions that could indicate mood, anxiety, depression, agitation, and fatigue, as well as measures of the patient's activity. Similarly, cognitive parameters can be determined from tests administered on a smart device. Physiological parameters and sleep parameters can be measured at the portable monitoring systems 206 and 208. Motor and biomarker parameters can be measured in a clinical environment and recorded in the EHR database 204 for later retrieval, as can any of the physiological, sleep, behavioral, cognitive, and psychosocial parameters that cannot be measured using the portable monitoring device.

The central server 210 can be implemented as a dedicated physical server or as part of a cloud server arrangement. In addition to the remote server, data can be analyzed, in whole or in part, on the local device itself and/or in a federated learning mechanism, in which case, any data from the EHR 204 can be provided to the local device via an appropriate network interface. The central server 210 includes a parameter interface 214 that receives the data collected by the portable monitoring devices 206 and 208 and retrieved from the EHR system 204 via the network interface 212. The parameter interface 214 can provide the received data to a feature extractor 216 that extracts a plurality of parameters from the received data.

The feature extractor 216 determines a plurality of parameters representing the wellness-relevant parameters. In one example, the parameters can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. The plurality of parameters can also include parameters represent departures of the user from an established pattern in the received data. For example, values of a given parameter can be tracked over time, and measures of central tendency can be established, either overall or for particular time periods. The collected parameters can represent a departure of a given parameter from the measure of central tendency. For example, changes in the activity level of the user, measured by either or both of kinematic sensors and global positioning system (GPS) tracking can be used as a parameter. Additional elements of monitoring can include the monitoring of the user's compliance with the use of a smart phone, TV, portable device, a portable device. For example, a user may be sent messages by the system inquiring on their wellness level, general mood, or the status of any other wellness-relevant parameter on the portable computing device. A measure of compliance can be determined according to the percentage of these messages to which the user responds via the user interface on the portable computing device during a given time period.

In one implementation, the feature extractor 216 can perform a wavelet transform on a time series of values for one or more parameters to provide a set of wavelet coefficients. It will be appreciated that the wavelet transform used herein can be two-dimensional, such that the coefficients can be envisioned as a two-dimensional array across time and either frequency or scale. The feature extractor 216 can also include a facial expression classifier (not shown) that evaluates recorded data from a camera and/or recorded images or videos of the user's face from one of the portable monitoring devices 206 and 208, such as a smartphone or other mobile device, to assign an emotional state to the user at various times throughout the day. The extracted parameters can be categorical, representing the most likely emotional state of the user, or continuous, for example, as a time series of probability values for various emotional states (e.g., anxiety, discomfort, anger, etc.) as determined by the facial expression classifier. The feature extractor 216 can also include one or more image classifiers that reduce provided medical images retrieved from the EHR database 204 to categorical or continuous parameters. It will be appreciated that each of the facial expression classifier and the one or more image classifiers can be implemented using one or more of the models discussed below for use in the global model 222 or the personalized model 224. In one example, the feature extractor 216 can provide the medical images to a convolutional neural network (CNN) and extract one or more latent values from the CNN as parameters.

It will be appreciated that the specific parameters and features used for screening can vary with the implementation. For example, screening for pain may focus on measured autonomic parameters, sleep parameters, data from the patient's medical record, functional brain imaging, and self-reporting from the patient. In another example, screening for addiction, PTSD, phobias, panic disorders, anxiety, depression, and schizophrenia may focus on autonomic, behavioral, cognitive and psychosocial parameters, data from the patient's medical records, and self-reporting from the patient on various questionnaires. Screening for dementia may focus on brain and eye imaging, autonomic, psychosocial, sensory, biomarker, and cognitive parameters, and data from the patient's medical record. The patient's response, both self-reported and in the form of measured physiological values, to cues appropriate to their disorder can also be recorded and used for generating features, particularly for addiction, depression, PTSD, anxiety, and phobias. Screening for movement disorders such as Parkinson's disease (PD) and Huntington's disease, or for the aftereffects of strokes can focus on motor, autonomic, psychosocial, and cognitive parameters. It will be appreciated that the methods herein focus on monitoring and improving the behavioral and cognitive and autonomic nervous system aspects of these diseases with our approach. As a specific example, our approach helping to improve visuospatial impairments in Parkinson's disease such as visual perception, blurry vision, judging distances, depth perception, and other visual and spatial deficits that impact so many PD patients. Screening for obsessive compulsive disorder and neurodevelopment disorders, such as autism and autism related disorders, can focus on autonomic, psychosocial, and cognitive parameters. A feature selector 218 can select a subset of the plurality of parameters [0056] for use at the personalized model 224. In addition to the parameters provided at the feature extractor 216, the plurality of parameters can also include the outputs of another set of personalized and global models, with the predictions of one of those models used as an input to predict a parameter at the personalized model 224 or the global model 222. The feature selector 218 can select a subset of the plurality of features that provides a subset of the plurality of features that provides a best performance for the personalized model. It will be appreciated that this is determined from data gathered from the subject, and that the subset of the plurality of features can be selected both during an initial training and tuning of the personalized model 224 as well as at periodic intervals during operation of the model. In one implementation, the feature selector 218 is implemented as a generative adversarial model comprising a generative model that generates synthetic test samples and a discriminative model that attempts to distinguish the synthetic test samples from test samples taken from the data from the patient. In this implementation, the features associated with the discriminative model having a highest fitness value when trained against a generative model in the generative adversarial model are selected. In another implementation, a genetic algorithm can be employed to find a set of features having a highest fitness value. It will be appreciated that the fitness value can be calculated from values representing the accuracy of the classification, including both specificity and sensitivity where appropriate (e.g., for detection of a specific disorder). In some implementations, the calculation of the fitness value can also include other values representing the number or complexity of the features selected by the model.

In one example, the feature selector 218 is implemented as a variational autoencoder, a deep learning model with two main components an encoder and a decoder. A Variational Autoencoder (VAE) is an unsupervised learning model that learns a lower-dimensional latent representation of data and aims to reconstruct the original data from this representation. VAEs are particularly useful for anomaly detection because they are trained to capture the underlying structure of normal data, making it easier to identify unusual data points by comparing reconstruction errors. High reconstruction errors indicate that the model struggled to replicate certain data points, signaling potential anomalies.

In a standard autoencoder, the encoder directly outputs the latent representation. In a VAE, the encoder outputs the parameters (mean and log-variance) of a probability distribution in the latent space, instead of the latent representation itself. During training, the model learns to encode the input into the parameters of this latent distribution, rather than directly into a fixed latent vector. The sampling function then samples from this learned latent distribution, using the mean and log-variance outputs from the encoder. This reparameterization trick allows the gradients to be backpropagated through the sampling operation, enabling end-to-end training of the VAE model. The intuition is that by learning a distribution in the latent space, rather than a fixed representation, the VAE can better capture the underlying structure and variability of the input data, which can lead to improved performance in anomaly detection

The sampling function is implemented as a custom layer that is used as part of a variational autoencoder model to sample from the latent space distribution during the training and inference process. The function takes two argument representing the mean and log-variance of the latent space distribution. The function determines the batch size (batch) and the dimensionality of the latent space (dim) and generates a random Gaussian noise tensor epsilon with the same shape as the latent space, defined as the product of the batch size and the latent dimension. The decoder reconstructs the original input data from the latent representation. The final output layer uses a linear activation function to generate values that closely resemble the input data. In one example, the variational autoencoder is compiled using the Adam optimizer with Mean Squared Error (MSE) as the loss function.

Each of the global model 222 and the personalized model 224 can utilize one or more pattern recognition algorithms, each of which analyze a subset of the extracted parameters to assign a continuous or categorical clinical parameter to the user. The models can utilize one or more pattern recognition algorithms, each of which analyze the extracted features or a subset of the extracted features to assign a continuous or categorical clinical parameter to the user representing the likelihood that the patient would benefit from focused ultrasound treatment. In one example, the clinical parameter can be a continuous parameter representing an overall wellness of the user, the likelihood that the patient has a specific disorder, such as Alzheimer's disease, the likelihood that a patient will benefit from treatment generally or from a specific treatment, such as focused ultrasound treatment, given a known diagnosis, or a likelihood that the patient will begin experiencing symptoms associated with a disorder. In another example, the clinical parameter can be a categorical parameter representing whether the patient has a disorder, categories representing changes in symptoms associated with a disease or disorder (e.g., “improving”, “stable, “worsening”), categories representing a predicted response to treatment generally or a specific treatment, whether the patient has a specific disorder, the severity of a disorder, or categories representing ranges of likelihoods that the patient falls into one of these categories.

Where multiple classification or regression algorithms are used in the global model 222 and the personalized model 224, an arbitration element can be utilized to provide a coherent result from the plurality of algorithms. The training process of a given classifier will vary with its implementation, but training generally involves a statistical aggregation of training data into one or more parameters associated with the output class. Each training sample comprises a plurality of input parameters and a label that represents the correct output from the system. The training process can be accomplished on a remote system and/or on the local device or wearable, app. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

Federated learning (aka collaborative learning) is a predictive technique that trains an algorithm across multiple decentralized edge devices or servers holding local data samples, without exchanging their data samples. This approach stands in contrast to traditional centralized predictive techniques where all data samples are uploaded to one server, as well as to more classical decentralized approaches which assume that local data samples are identically distributed. Federated learning enables multiple actors to build a common, robust predictive model without sharing data, thus addressing critical issues such as data privacy, data security, data access rights, and access to heterogeneous data. Its applications are spread over a number of industries including defense, telecommunications, IoT, or pharmaceutics.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. Another example is utilizing an autoencoder to detect outlier in wellness-relevant parameters as an anomaly detector to identify when various parameters are outside their normal range for an individual.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

A model selector 226 receives a third subset of the plurality of parameters and selects one of the global model 222 and the personalized model 224 according to the third subset of the plurality of parameters. It will be appreciated that, depending on the implementation, the model selector 226 can select an output of the two models or select only one of the two models to be active for a given period of time. In one example, the model selector 226 reviews the third subset of the plurality of parameters to determine if the times series associated with the third subset of the plurality parameters contain high quality data. In one example, the model selector 226 includes a rule-based expert system that evaluates the time series of values for each of the third subset of the plurality of parameters to select between the global model 222 and the personalized model 224. In particular, the model selector 226 can evaluate the data for missing data, data outside of a plausible range, or metadata provided by a sensor indicating possible errors in the portable monitoring devices 206 and 208. The third subset of the plurality of parameters can be a subset of the first or second subset of parameters, such that the model selector 226 evaluates the quality of at least some of data provided to either the global model 222 or the personalized model 224. In one example, the third subset of the plurality of parameters is a subset of the second subset of parameters, such that the model selector 226 evaluates the quality of data provided to the personalized model 224, using the personalized model when high quality data is available and defaulting to the global model 222 when it is not. In this example, the model selector 224 calculates a data quality metric, based on the completeness (e.g., lack of missing values) and plausibility (e.g., lack of data outside of expected ranges or data with an unlikely range and rate of variability), and compares the metric to a threshold value to determine if the personalized model 224 or the global model 222 can be used.

A feedback component 228 can tune various parameters of the global model 222 and the personalized model 224 based upon the accuracy of predictions made by the models. Parameters associated with the models, such as thresholds for producing categorical inputs or outputs from continuous values or parameters associated with the pattern recognition algorithms comprising the models, can be adjusted according to the differences in the actual and predicted outcomes. In one implementation, a reinforcement learning approach can be used to adjust the model parameters based on the accuracy of either predicted future values of wellness-relevant parameters at intermediate stages of the models or the output of the models. For example, a decision threshold used to generate a categorical output from a continuous index produced by the either of the global model 222 and the personalized model 224 can be set at an initial value based on feedback from a plurality of models from previous users and adjusted via the reinforcement model to generate a decision threshold specific to the user. In addition to changing the model parameters, the feature selector 218 can alter the features used in the personalized model based on new training data generated from measured outcomes for the subject, and the model selector 226 can alter any threshold for a quality metric used for selecting between the personalized model 224 and the global model 222 based on this new training data.

In one implementation, the global model 222 and the personalized model 224 can include a constituent model that predicts future values for the wellness-related parameters or aggregate parameters, such as a convolutional neural network that is provided with one or more two-dimensional arrays of wavelet transform coefficients as an input. The wavelet coefficients detect changes not only in time, but also in temporal patterns, and can thus reflect changes in the ordinary biological rhythms of the user. Additionally, or alternatively, the predictive model can use constituent models that predict current or future values for the wellness-related or aggregate parameters, with these measures then used as features for generating the output of the predictive model. This data can also be used to group the user with users who respond similarly to these parameters, with data fed back from users within a given group used to better tailor the model to the user.

The global model 222 and the personalized model 224 can also be used to facilitate a feedback strategy to one or more designated recipients, which can include the user, a health care provider, family, friends, social system, a care team, a supervisor, a coach, a caretaker, and other entities, in which a course of action is suggested, for example, in response to a change in the wellness of the individual. Examples of disorders for which detection or the disorder or a heightened risk of the disorder can trigger intervention include, but are not limited to, anxiety, depression, suicidal thoughts, stress, mood, agitation, obsessions, compulsion, OCD, Parkinson's, tremor, and chronic pain. In one implementation, the value produced by the selected one the global model 222 and the personalized model 224 can represent a probability of an onset of symptoms associated with a disorder within a predetermined time window, with an intervention triggered at an intervention component 230 when the value exceeds a threshold value. It will be appreciated that the threshold for an intervention can be based on the triggering disorder and the length of the time window, with more extensive interventions and predictions over longer time periods requiring higher thresholds for intervention.

Further, prediction at different intervention windows can be performed with different sets of models, even for the same disorder. For example, prediction of substance use within the next day could be performed with a first set of a global model and personalized model, while a prediction of substance use within three days can use a second set of a global model and a personalized model with different parameters and input features that the first set. In addition, where the output of a set of models is used at a downstream set of models, the output of the upstream model can be used in addition to that of the downstream model to trigger an intervention. For example, a model predicting anxiety levels might contribute to a model predicting substance use. In this example, a heightened level of anxiety might trigger an intervention to reduce anxiety, and thus avoid substance use, even when the substance use model would not trigger an intervention. Some interventions can be provided based on more straightforward rule-based inputs. For example, tips on increasing physical activity can be provided based upon an increase in measured weight or a decrease in measured movement, regardless of the output of the selected one of the global model 222 and the personalized model 224.

The intervention component 230 can, for example, provide feedback to the [0072] user at a portable monitoring device (e.g., 208), such as a cellular phone or tablet, from the system for raising awareness of a detected issue or education on that issue or providing a suggested course of action, provide a message to a supervising physician to recommend initiating a treatment or modifying an existing treatment including but not limited to focused ultrasound treatment, medications, biologicals, surgical intervention, behavioral and social intervention, a digital intervention provided to the portable monitoring device, notifying a care provider to contact the individual, directing an individual to go to a clinic, support group, emergency room, or hospital with provided directions or a location for the clinic, support group, emergency room, or hospital, or directing the user to obtain additional testing.

It will be appreciated that a suggested course of action can be any course of action intended to enhance the wellness of the user or others and can include, for example, taking a prescribed meditation, performance of prescribed exercises, cessation of a current activity, and contacting a medical professional. In one example, when the user is determined to be experiencing significant stress, the feedback can include a “digital reset” in which the user is instructed to engage in deep breathing or other stress reduction techniques. If this is ineffective or if the problem reoccurs, the feedback can be elevated to a digital intervention prescribed by a medical expert, in which the user engages in guided stress reduction techniques as periodic intervals. If this is also ineffective, the user can be instructed to seek the assistance of a counselor, coach, or therapist. It will be appreciated that the model is predictive, and thus interventions for stress, pain, and similar issues can be suggested before the user is even aware of the issue. In another example, a user can be instructed in sleep hygiene in response to indications of inadequate or restless sleep, directed to engage in a sleep study, or referred to a physician for analysis. It will be appreciated that interventions associated with sleep can also be assigned for other detected issues, such as decreased cognitive function, decreased athletic, job, or other performance, or heightened risks of stroke and heart disease.

The intervention component 230 can also provide feedback based on the user's compliance with courses of action suggested by the system, interaction with one or more applications on the portable monitoring devices 206 and 208 for data gathering (e.g., questionnaires or cognitive tests), or for improvement in overall health metrics. For example, the intervention component 230 can award a digital badge for the user's profile based on completion of various objectives. Alternatively, a point system can be used, with the user provided with points for completing various objectives, with the points exchangeable for various rewards.

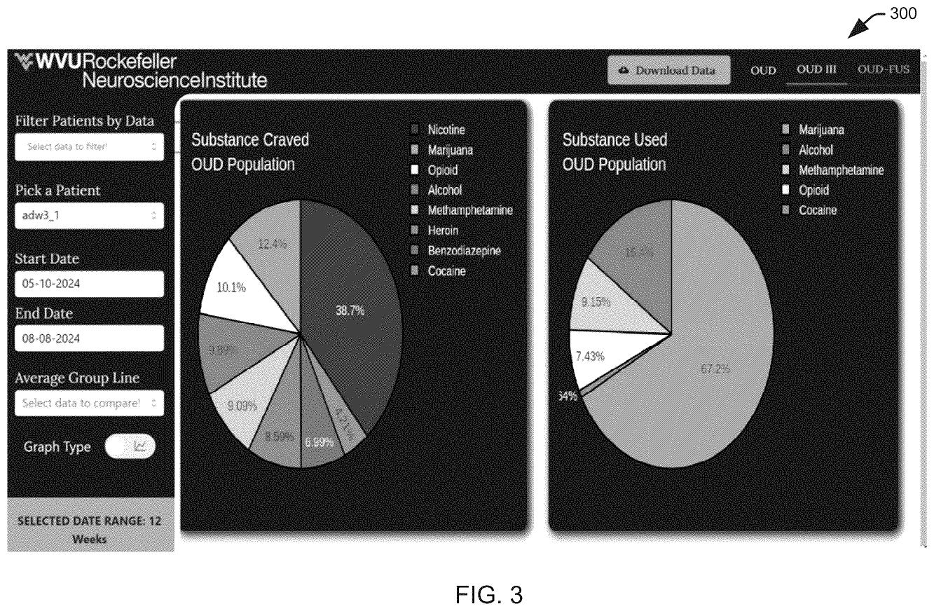

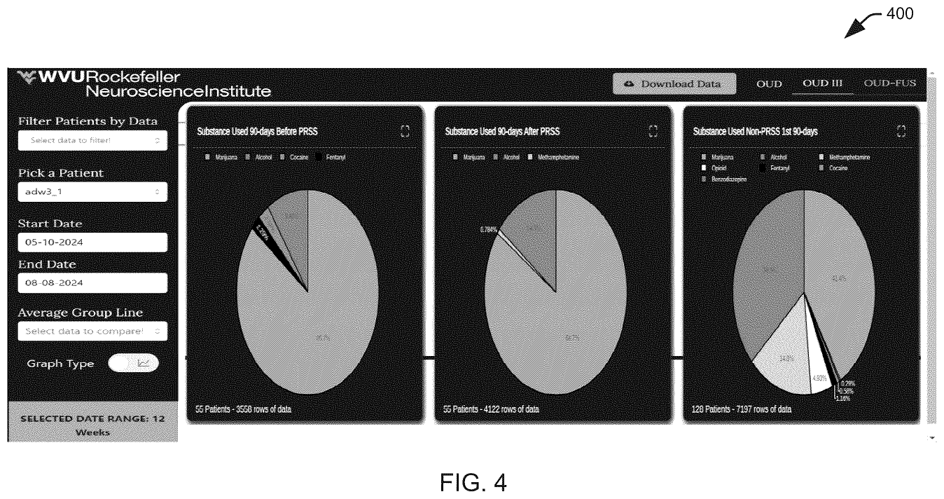

The system can also include a physician dashboard 232 that provides a user interface to allow a supervising physician or other medical professional to review data for patients and set individual goals and thresholds for patients for various metrics. FIG. 3 is a first screenshot 300 from the physician dashboard 232 illustrating data for a population of patients. In the illustrated screenshot, two charts are provided, representing craved and used substances for a population of patients being treated for opioid use disorder (OUD). FIG. 4 is a second screenshot 400 from the physician dashboard 232 illustrating data for a population of patients. In the illustrated screenshot, three charts are provided, representing substance use in ninety days prior to meeting with a peer recovery support specialist, substance use for ninety after prior to meeting with a peer recovery support specialist craved, and substance use for patients not meeting with a peer recovery support specialist for a population of patients being treated for OUD.

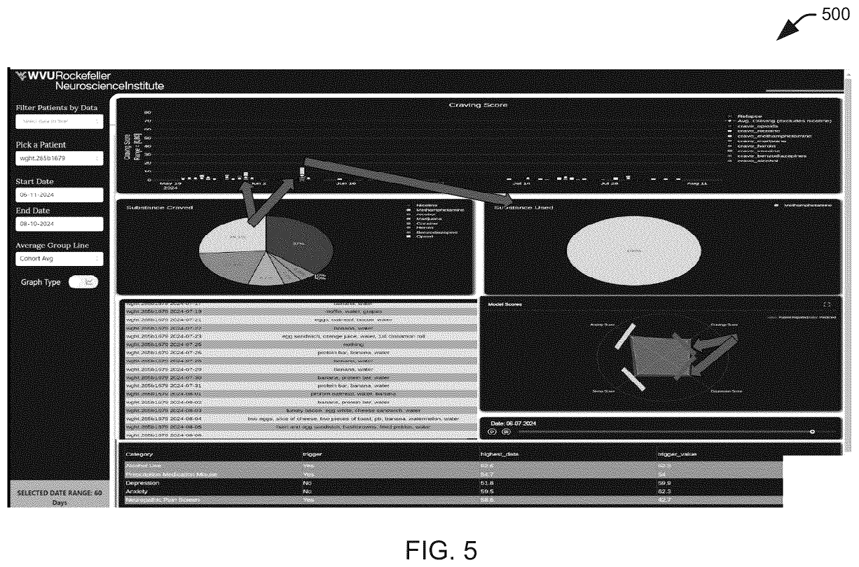

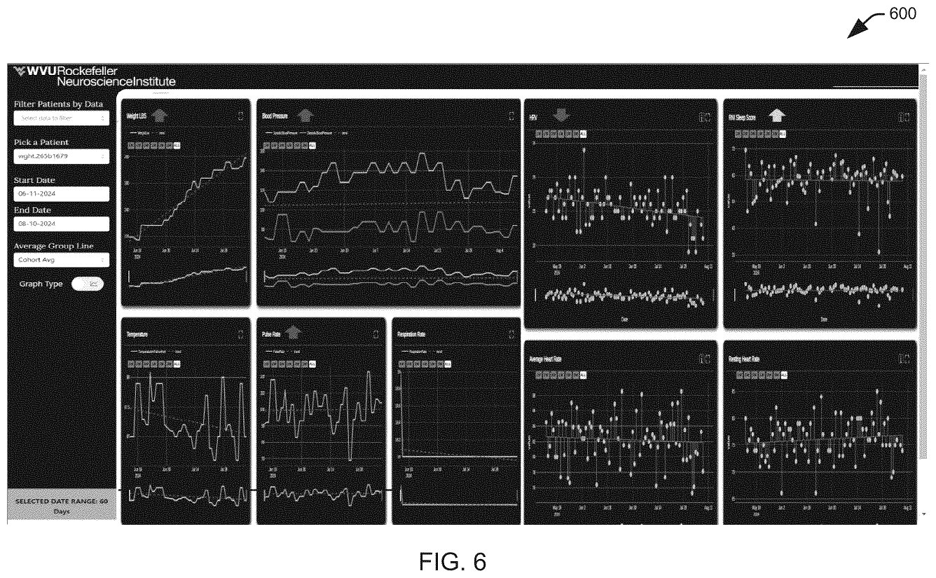

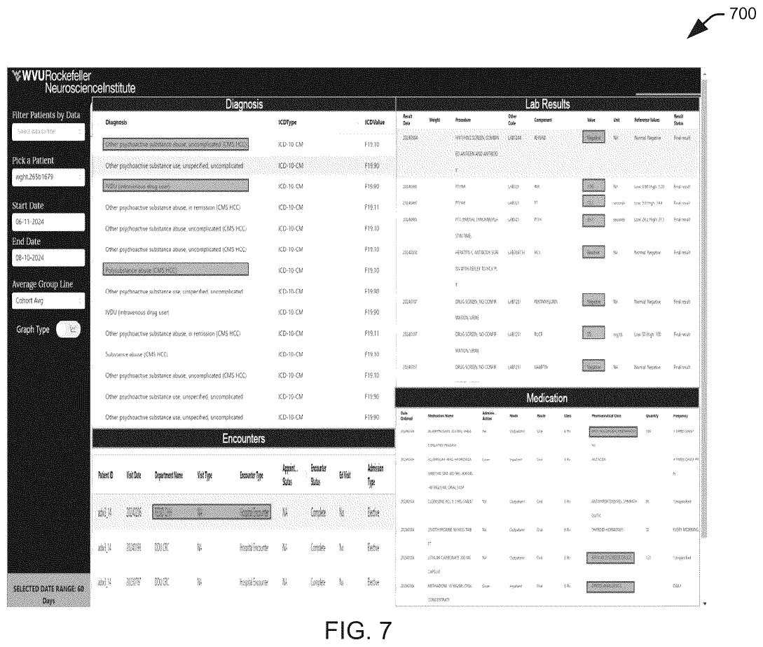

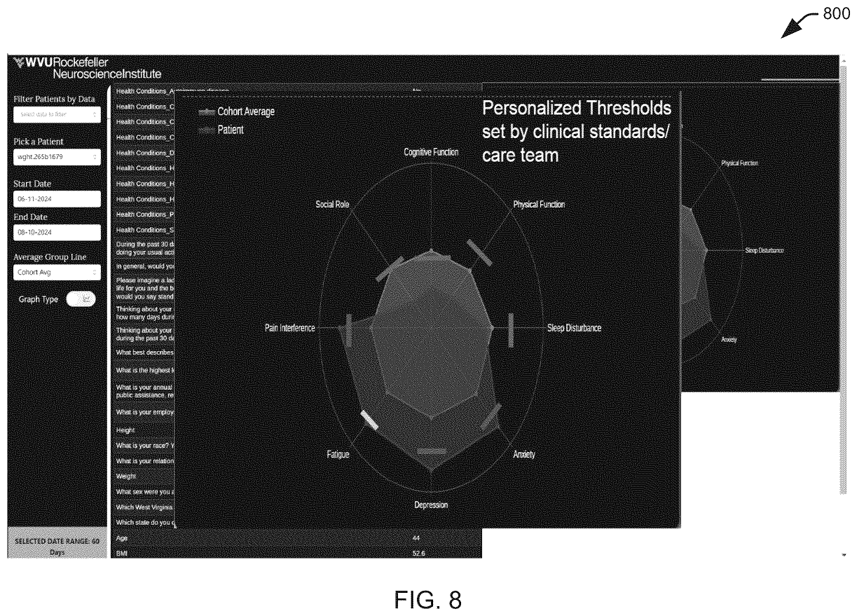

FIG. 5 is a first screenshot 500 from the physician dashboard 232 illustrating data for an individual patient. This screenshot 500 shows cravings and substances used for an individual patient being treated for OUD, as well as data collected from an electronic health records system and from the portable monitoring devices. FIG. 6 is a second screenshot 600 from the physician dashboard 232 illustrating data for an individual patient. The various charts track monitored wellness-related parameters for the patient over time to allow the physician to review trends in these values. FIG. 7 is a third screenshot 700 from the physician dashboard 232 illustrating data for an individual patient. In this screen, a medical record for the patient is provided, with various diagnoses, test results, encounters with medical professionals, and prescribed medications. FIG. 8 is a screenshot for an interface in the physician dashboard 232 that allows a physician to selecting personalized thresholds for a patient for various metrics provided by instantiations of the global model 222 and the personalized model 224. In this instance, eight separate instantiations of the models 222 and 224 are used to provide eight separate metrics representing the wellness of the user, and the physician can select thresholds for each metric that will trigger an alert or intervention. It will be appreciated that these personalized thresholds can be used in addition to automated thresholds generated at the model for providing interventions or in place of these thresholds.

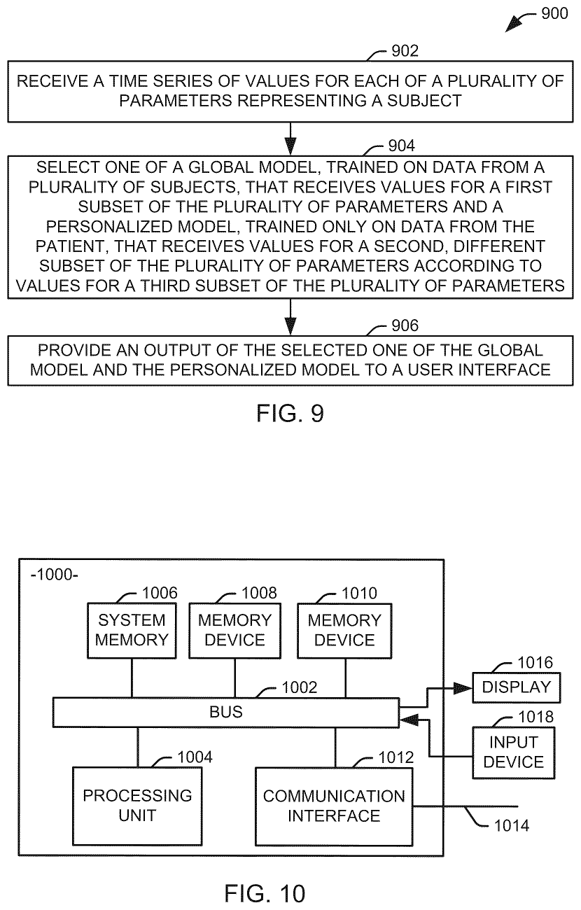

In view of the foregoing structural and functional features described above, example methods will be better appreciated with reference to FIG. 9. While, for purposes of simplicity of explanation, the example methods of FIG. 9 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

FIG. 9 illustrates a method 900 for monitoring a wellness of a user. At 902, a time series of values are received for each of a plurality of parameters representing a subject. For example, a time series of values for each of the plurality of parameters can be received from one or more of an electronic health records system and a portable device carried or worn by the subject via a network interface. At 904, either a global model, trained on data from a plurality of subjects, or a personalized model, trained only on data from the patient, is selected. The global model receives the time series of values for each of a first subset of the plurality of parameters and a personalized model, and the personalized model receives the time series of values for each of a second, different subset of the plurality of parameters. The selection of the models can be made according to the time series of values for each of a third subset of the plurality of parameters. The second subset of the plurality of parameters can be personalized for the patient by selecting the parameters according to data collected for the patient. In one example, the third subset of the plurality of parameters is a proper subset of the second subset of the plurality of parameters.

At 906, an output of the selected one of the global model and the personalized model is provided to a user interface. In one example, the output of the selected one of the global model and the personalized model comprises a value representing a likelihood that the subject will experience symptoms associated with a condition within a predetermined period of time. In this example, feedback can be provided to the subject at a portable device via the network interface if the output of the selected one of the global model and the personalized model exceeds a threshold value. For example, the provided feedback can be a digital therapy intended to mitigate the symptoms associated with the condition.

FIG. 10 is a schematic block diagram illustrating an exemplary system 1000 of hardware components capable of implementing examples of the systems and methods disclosed in FIGS. 1-9. The system 1000 can include various systems and subsystems. The system 1000 can be a personal computer, a laptop computer, a workstation, a computer system, an appliance, an application-specific integrated circuit (ASIC), a server, a server blade center, a server farm, etc.

The system 1000 can includes a system bus 1002, a processing unit 1004, a system memory 1006, memory devices 1008 and 1010, a communication interface 1012 (e.g., a network interface), a communication link 1014, a display 1016 (e.g., a video screen), and an input device 1018 (e.g., a keyboard and/or a mouse). The system bus 1002 can be in communication with the processing unit 1004 and the system memory 1006. The additional memory devices 1008 and 1010, such as a hard disk drive, server, stand-alone database, or other non-volatile memory, can also be in communication with the system bus 1002. The system bus 1002 interconnects the processing unit 1004, the memory devices 1006-1010, the communication interface 1012, the display 1016, and the input device 1018. In some examples, the system bus 1002 also interconnects an additional port (not shown), such as a universal serial bus (USB) port.

The processing unit 1004 can be a computing device and can include an application-specific integrated circuit (ASIC). The processing unit 1004 executes a set of instructions to implement the operations of examples disclosed herein. The processing unit can include a processing core.

The additional memory devices 1006, 1008, and 1010 can store data, programs, instructions, database queries in text or compiled form, and any other information that can be needed to operate a computer. The memories 1006, 1008 and 1010 can be implemented as computer-readable media (integrated or removable) such as a memory card, disk drive, compact disk (CD), or server accessible over a network. In certain examples, the memories 1006, 1008 and 1010 can comprise text, images, video, and/or audio, portions of which can be available in formats comprehensible to human beings. Additionally or alternatively, the system 1000 can access an external data source or query source through the communication interface 1012, which can communicate with the system bus 1002 and the communication link 1014.

In operation, the system 1000 can be used to implement one or more parts of a wellness monitoring system in accordance with the present invention. Computer executable logic for implementing the wellness monitoring system resides on one or more of the system memory 1006, and the memory devices 1008, 1010 in accordance with certain examples. The processing unit 1004 executes one or more computer executable instructions originating from the system memory 1006 and the memory devices 1008 and 1010. The term “computer readable medium” as used herein refers to any medium that participates in providing instructions to the processing unit 1004 for execution, and it will be appreciated that a computer readable medium can include multiple computer readable media each operatively connected to the processing unit.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

What is claimed is:

1. A system comprising:
a processor; and
a non-transitory computer-readable medium storing executable instructions that are executable by the processor, the executable instructions comprising:
a parameter interface that receives a time series of values for each of a plurality of parameters representing a subject;
a global model, trained on data from a plurality of subjects, that receives the time series of values for each of a first subset of the plurality of parameters;
a personalized model, trained only on data from the subject, that receives the time series of values for each of a second subset of the plurality of parameters, the first subset of the plurality of parameters being different from the second subset of the plurality of parameters; and
a model selector that selects one of the global model and the personalized model according to the time series of values for each of a third subset of the plurality of parameters, an output of the selected one of the global model and the personalized model being provided to a subject interface.

2. The system of claim 1, wherein the third subset of the plurality of parameters is a subset of the second subset of the plurality of parameters.

3. The system of claim 1, wherein the global model is a first global model representing a first condition, the plurality of subjects is a first plurality of subjects, the personalized model is a first personalized model representing the first condition, and the model selector is a first model selector, the system further comprising:
a second global model, trained on a data from a second plurality of subjects and representing a second condition, that receives the time series of values for each of a fourth subset of the plurality of parameters;
a second personalized model, trained only on data from the subject and representing a second condition, that receives the time series of values for each of a fifth subset of the plurality of parameters, the fourth subset of the plurality of parameters being different from the fifth subset of the plurality of parameters; and
a second model selector that selects one of the second global model and the second personalized model according to the time series of values for each of a sixth subset of the plurality of parameters, an output of the selected one of the second global model and the second personalized model being provided to a subject interface.

4. The system of claim 3, wherein the first subset of the plurality of parameters is different than the fourth subset of the plurality of parameters and the second subset of the plurality of parameters is different than the fifth subset of the plurality of parameters.

5. The system of claim 3, wherein the first model selector operates independently of the second model selector, such that, at a given point in time, the first model selector can select the first personalized model while the second model selector selects the second global model.

6. The system of claim 1, further comprising a network interface that provides the time series of values for each of the plurality of parameters representing the subject to the parameter interface, the network interface communicating with one of an electronic health records system and a portable device carried or worn by the subject via an Internet connection.

7. The system of claim 6, the network interface communicating with each of the electronic health records system and the portable device via the Internet connection.

8. The system of claim 1, further comprising a feature selector that selects the second subset of the plurality of parameters for the personalized model from the data from the patient.

9. The system of claim 8, wherein the feature selector comprises a generative adversarial network comprising a generative model that generates synthetic test samples, and a discriminative model that attempts to distinguish the synthetic test samples from test samples taken from the data from the patient.

10. The system of claim 1, wherein the model selector comprises a rule-based expert system that evaluates the time series of values for each of the third subset of the plurality of parameters to select between the global model and the personalized model.

11. The system of claim 1, wherein each of the global model and the personalized model are implemented as recurrent neural networks.

12. The system of claim 1, wherein the output of the selected one of the global model and the personalized model comprises a value representing a likelihood that the subject will experience symptoms associated with a condition within a predetermined period of time.

13. A method comprising:
receiving a time series of values for each of a plurality of parameters representing a subject;
selecting one of a global model, trained on data from a plurality of subjects, that receives the time series of values for each of a first subset of the plurality of parameters and a personalized model, trained only on data from the patient, that receives the time series of values for each of a second subset of the plurality of parameters according to the time series of values for each of a third subset of the plurality of parameters, the first subset of the plurality of parameters being different from the second subset of the plurality of parameters; and
providing an output of the selected one of the global model and the personalized model to a user interface.

14. The method of claim 12, wherein receiving the time series of values for each of the plurality of parameters representing the subject further comprises receiving the time series of values for each of the plurality of parameters from one of an electronic health records system and a portable device carried or worn by the subject via a network interface.

15. The method of claim 12, further comprising selecting the second subset of the plurality of parameters for the personalized model according to the data from the patient.

16. The method of claim 12, wherein the third subset of the plurality of parameters is a proper subset of the second subset of the plurality of parameters.

17. The method of claim 12, wherein the output of the selected one of the global model and the personalized model comprises a value representing a likelihood that the subject will experience symptoms associated with a condition within a predetermined period of time, the method further comprising providing feedback to the subject at a portable device via the network interface if the output of the selected one of the global model and the personalized model exceeds a threshold value.

18. The method of claim 17, wherein the provided feedback is a digital therapy intended to mitigate the symptoms associated with the condition.

19. A system comprising:
a processor;
a network interface; and
a non-transitory computer-readable medium storing executable instructions that are executable by the processor, the executable instructions comprising:
a parameter interface that receives a time series of values for each of a plurality of parameters representing a subject through an Internet connection via the network interface;
a first recurrent neural network, trained on a plurality of training samples from a plurality of subjects, each of the training samples comprising a value representing an outcome for a subject of the plurality of subjects and a time series of values for each of a first subset of the plurality of parameters;
a second recurrent neural network, trained on a plurality of training samples from the subject, each of the training samples comprising a value representing an outcome for the subject and a time series of values for each of a second subset of the plurality of parameters, the first subset of the plurality of parameters being different from the second subset of the plurality of parameters; and
a model selector that selects one of the first recurrent neural network and the second recurrent neural network according to the time series of values for each of a third subset of the plurality of parameters, provides the time series of values for a subset of the plurality of parameters to the selected one of the first recurrent neural network and the second recurrent neural network as inputs, and provides an output of the selected one of the first recurrent neural network and the second recurrent neural network to a user interface, the output of the selected one of the first recurrent neural network and the second recurrent neural network comprising a value representing a likelihood that the subject will experience symptoms associated with a condition within a predetermined period of time.

20. The system of claim 16, further comprising a feature selector that selects the second subset of the plurality of parameters for the personalized model from the data from the patient, the feature selector comprising a generative adversarial network comprising a generative model that generates synthetic test samples, and a discriminative model that attempts to distinguish the synthetic test samples from test samples taken from the data from the patient.
